(19) United States
(12) Patent Application Publication
(10) Pub. No.: US 2015/0342985 A1
(43) Pub. Date: Dec. 3, 2015

(54) COMPOSITIONS COMPRISING LITHIUM

(71) Applicant: Avi FRIEDLICH, Quincy, MA (US)

(72) Inventor: Avi Friedlich, Quincy, MA (US)

(21) Appl. No.: 14/758,020

(22) PCT Filed: Dec. 27, 2013
(86) PCT No.: PCT/US13/77988

§ 371 Date: Jun. 26, 2015

Related U.S. Application Data
(60) Provisional application No. 61/747,097, filed on Dec. 28, 2012.

(51) Int. Cl. A61K 33/00; A61K 31/513; A61K 33/14

(52) U.S. Cl. CPC A61K 33/00; A61K 33/14; A61K 31/513

(57) ABSTRACT

This invention relates to compositions comprising lithium and methods of their use.

### CLAIM OF PRIORITY

This application claims priority to U.S. Ser. No. 61/747,097, filed Dec. 28, 2012, the entire contents of which is incorporated herein by reference.

### FIELD OF INVENTION

This invention relates to compositions comprising lithium and methods of their use.

### BACKGROUND OF INVENTION

Lithium is unique for its chemistry as the lightest ion in solution. It is an abundant element on the earth's surface, and is also found in living organisms and tissues, including the human body and the human brain. Because only trace levels of lithium are present in the brain, only recently has it become possible to overcome technical obstacles to measure lithium levels accurately and study its effects in biological systems. As a result, research into the normal biology of Lithium is in its infancy. Meanwhile, Lithium has been grandfathered into Medicine and Psychiatry, established over 50 years ago as a therapy, and to this day, it stands alone as the “gold standard” for managing psychiatric symptoms in many psychiatric conditions, including mood disorders (e.g., Bipolar disorder and Major depressive disorder, and psychotic disorders (e.g., schizophrenia and schizoaffective disorders)). The present invention is the first to suggest that the current formulation of lithium may be sub-optimal for patients and that it can be improved upon, and rapidly so.

In nature, elements may be characterized according to the properties of their stable isotopes. For lithium, there are two stable isotopes in nature, referred to as, 6-Li and 7-Li. The isoform 7-Li is about 12.5 times more abundant in the environment, and the two isotopes have much different chemistries. For example, they fractionate readily during weathering and mineral formation, and show differential inorganic properties. It is also well known that the lithium isotopes behave differently from each other in the human body.

### SUMMARY OF INVENTION

In the most general sense, the present invention is based on the discovery that there may exist a disequilibrium in the lithium isotope ratios between the brain and the external environment. Although this had never been reported for lithium, for other elements such as iron, it is well established that the body is capable of partitioning stable isotopes of other elements.

The invention is founded more specifically on the discovery that partitioning of the lithium isotopes may be a biologically regulated phenomenon, subject to change in neuropsychiatric disease states, and precisely, that an elevated 6-Li to 7-Li ratio is specific to the pathophysiology of bipolar disorder.

This suggests that the disease mechanism itself for bipolar disorder may relate to the neurobiology of lithium, and moreover, that 6-Li may be more effective than 7-Li as a medicine. Current pharmaceutical formulations of the stable lithium isotopes are not separated in current pharmaceutical formulations, whereas in the present invention, they are.

In one aspect, the invention features a pharmaceutical composition comprising lithium, wherein the lithium comprises at least 8% 6-Li (e.g., at least 10% 6-Li, at least 20% 6-Li, at least 50% 6-Li, at least 70% 6-Li, at least 80% 6-Li, at least 90% 6-Li, or at least 99% 6-Li); and wherein the composition is formulated for immediate or extended release.

In some embodiments, the composition is in the form of a tablet or capsule. In some embodiments, the composition is formulated as a salt, for example, a carbonate, a chloride an orotate, aspartate, amide, acetate, arachidonate, adrenate, bromide, chloride, citrate, ethylester, glutamate lithium, glycolate, gluconate, glucoheptanate glycerides, diglyceride, triglyceride lithium methylmethacrylate orotate, succinate, phenylalanine, lauryl sulphate, linoleate, alpha-linoleate. gamma-linolenate, docosahexaenoate, eicosapentaenoate, ethylenediaminetetraacetate, docosahexaenoate or dihomogammalinolenic acid.

In some embodiments, the composition is the composition is formulated for administration once, twice or three times per day or more, for at least 5 days, at least 6 days, at least 7 days, at least one month, or at least one year or longer. In some embodiments, the composition comprises at least 25 mg, at least 50 mg, at least 100 mg, at least 150 mg, at least 200 mg, at least 250 mg, at least 300 mg, at least 350 mg, at least 400 mg, or at least 450 mg 6-Li. In some embodiments, the lithium comprises at least 8% 6-Li (e.g., at least 10% 6-Li, at least 20% 6-Li, at least 50% 6-Li, at least 70% 6-Li, at least 80% 6-Li, at least 90% 6-Li, or at least 99% 6-Li); and an additional therapeutic agent.

In one aspect, the invention features a method of treating a subject comprising administering a composition described herein.

In some embodiments, the composition is formulated for administration for at least 5 days, at least 6 days, at least 7 days, at least one month, or at least one year or longer. In some embodiments, the composition is the composition is formulated for administration once, twice or three times per day or more. In some embodiments, the composition comprises at least 50 mg, at least 100 mg, at least 150 mg, at least 200 mg, at least 250 mg, at least 300 mg, at least 350 mg, at least 400 mg, or at least 450 mg 6-Li. In some embodiments, the lithium comprises at least 8% 6-Li (e.g., at least 10% 6-Li, at least 20% 6-Li, at least 50% 6-Li, at least 70% 6-Li, at least 80% 6-Li, at least 90% 6-Li, or at least 99% 6-Li); and an additional therapeutic agent.

In one aspect, the invention features a method of treating a subject, the method comprising administering to the subject a pharmaceutical composition comprising lithium, wherein the lithium comprises at least 8% 6-Li (e.g., at least 10% 6-Li, at least 20% 6-Li, at least 50% 6-Li, at least 70% 6-Li, at least 80% 6-Li, at least 90% 6-Li, or at least 99% 6-Li) and an additional therapeutic agent.

In one aspect, the invention features a method of treating a subject, the method comprising administering the subject a pharmaceutical composition comprising lithium, wherein the lithium comprises at least 8% 6-Li (e.g., at least 10% 6-Li, at least 20% 6-Li, at least 50% 6-Li, at least 70% 6-Li, at least 80% 6-Li, at least 90% 6-Li, or at least 99% 6-Li) at an amount sufficient to achieve a therapeutic effect, wherein the composition is administered to the subject for at least about 5 days at least 6 days, at least 7 days, at least one month, or at least one year or longer.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments featured in the invention are set forth in the accompanying drawings and the description below. Other features, objects, and advantages featured in the invention will be apparent from the description and drawings, and from the claims.

### BRIEF DESCRIPTION OF DRAWINGS

FIG. 1 is a graph of neocortical lithium levels in subjects with bipolar disorder (BD) or schizophrenia (Sz), and in normal controls (NC). Bars indicate the mean for each group, with each subject represented by an open circle. **P<0.01.

FIG. 2 is a graph of the relative deviation (δ) of 7-Li from environmental distribution in subjects with bipolar disorder (BD), bipolar disorder receiving lithium treatment at time of death (BD+ Li), schizophrenia (Sz) and normal controls (NC). Mean +/−S.E.M. ** p<0.05. The established 7-Li: 6-Li ratio in nature is 92.41%: 7.59%.

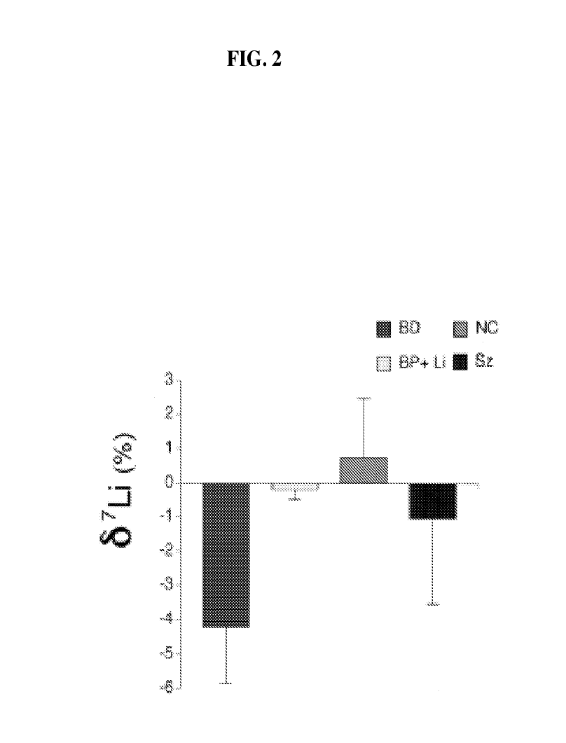

### DETAILED DESCRIPTION

The invention is based, at least in part, on the discovery that different isoforms of lithium are present in different amounts in brains of human subjects having various psychiatric disorders.

The lithium formulations currently given to patients generally include naturally occurring ratios of Li-7 to Li-6, for example about 93% Li-7: 7% Li-6. Applicants have surprisingly discovered that Li-6 is better able to penetrate the brain, and therefore pharmaceutical compositions that are enriched for Li-6 will have greater efficacy for treatment of psychiatric disorders such mood disorders. Because Li-6 is capable of penetrating the brain to a great extent, in some embodiments, compositions enriched for Li-6 can be administered at a lower dose, for example, relative to compositions that include naturally occurring ratios of Li-7 to Li-6.

### Lithium

Lithium exists in two stable isoforms, 6-Li and 7-Li. The invention provides formulations comprising lithium isoforms (e.g., 6-Li or 7-Li) in various ratios, for example, in ratios that are enriched for a single isoform relative to the naturally occurring ratios. In some embodiments, the formulation is enriched for Li-7 (e.g., includes greater than about 93% by wt. Li-7, greater than about 95%, greater than about 97% or greater than about 99%). In some embodiments, the formulation is enriched for Li-6 (e.g., includes great than about 7% by wt. Li-6, greater than about 10%, greater than about 15%, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95%, or about 100%).

In some embodiments, the lithium in the formulation is a salt of lithium. A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base carbonate, a chloride an orotate, aspartate, amide, acetate, arachidonate, adrenate, bromide, chloride, citrate, ethylester, glutamate lithium, glycolate, gluconate, glucoheptanate glycerides, diglyceride, triglyceride lithium methylmethacrylate orotate, succinate, phenylalanine, lauryl sulphate, linoleate, alpha-linoleate.gamma-linolenate, docosahexaenoate, eicosapentaenoate, ethylenediaminetetraacetate, docosahexaenoate or dihomogammalinolenic acid.

Acid addition salts include those derived from nontoxic inorganic acids, such as hydrochloric, nitric, phosphoric, sulfuric, hydrobromic, hydroiodic, and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like.

### Pharmaceutical Compositions

The pharmaceutical compositions featured in the invention include lithium, such as 6-Li and/or 7-Li. Typically, the composition will include at least 6% 6-Li, at least 10% 6-Li, at least 20% 6-Li, at least 50% 6-Li, at least 70% 6-Li, at least 80% 6-Li, at least 90% 6-Li, or at least 99% 6-Li.

In some embodiments, the composition will include at least 6% 7-Li, at least 10% 7-Li, at least 20% 7-Li, at least 50% 7-Li, at least 70% 7-Li, at least 80% 7-Li, at least 90% 7-Li, or at least 99% 7-Li.

A pharmaceutical composition containing 6-Li and/or 7-Li can be formulated for extended release, such as in the form of a tablet or capsule. “Lithibid” and “Eskalith” are tablets containing 300 mg and 450 mg, respectively, of lithium carbonate, for once a day administration. The formulation can be for administration for at least 5 days, at least 6 days, at least 7 days, at least one month, or at least one year or longer. The composition can be formulated for administration once, twice or three times per day or more.

An exemplary pharmaceutical composition comprises at least 25 mg, at least 50 mg, at least 100 mg, at least 150 mg, at least 200 mg, at least 250 mg, at least 300 mg, at least 350 mg, at least 400 mg, or at least 450 mg lithium, e.g., 6-Li and/or 7-Li. In some embodiments, the dose is such that it achieves pharmacoparameters of 6-Li that are measurable in blood.

Typically, a pharmaceutical composition includes a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible.

Pharmaceutical compositions may be in a variety of forms. These include, for example, liquid, semi-solid and solid dosage forms, such as liquid solutions (e.g., infusible solutions), dispersions or suspensions, tablets, pills, powders, liposomes and suppositories. The form can depend on the intended mode of administration and therapeutic application. Typically, compositions for the agents described herein are in the form of pills, capsule or oral solutions.

Pharmaceutical compositions typically must be sterile and stable under the conditions of manufacture and storage. A pharmaceutical composition can also be tested to insure it meets regulatory and industry standards for administration.

The composition can be formulated as a solution, microemulsion, dispersion, liposome, or other ordered structure suitable to high drug concentration. Generally, dispersions are prepared by incorporating an agent described herein into a sterile vehicle that contains a basic dispersion medium and the required other ingredients from those enumerated above. In the case of sterile powders for the preparation of sterile injectable solutions, typical methods of preparation are vacuum drying and freeze-drying that yields a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

### Treatment Methods and Administration

The lithium formulations described herein can be used to treat a subject, e.g., a subject in need of a therapeutic amount of Li, e.g., 6-Li. In some embodiments, the subject is treated with an amount of a composition described herein to achieve a therapeutic effect (e.g., to treat and/or ameliorate at least one symptom of a disorder described herein). In some embodiments, a subject is treated for at least about 1 week, at least about 2 weeks, at least about 3 weeks, at least about 4 weeks, etc. The physician adjusts the dose according to frequent assessments of clinical efficacy, blood levels, and possible side effects to achieve an optimal treatment plan.

The subject may have been previously diagnosed with a mood disorders (e.g., Bipolar disorder, Major depressive disorder, or a psychotic disorders (e.g. schizophrenia or schizoaffective disorders).

### Exemplary Second Accents

In some cases, the formulations described herein, e.g., formulations containing Li-6 or Li-7, include one or more additional agents, e.g., a second agent, or are administered in combination with a formulation containing a one or more additional agents. These additional agents may include a medication that falls in the category of an antidepressant, an antipsychotic or neuroleptic, or a mood stabilizer.

### Kits

Lithium formulations provided in the invention can be provided in a kit.

In one embodiment, the kit includes (a) a container that contains a composition that includes 6-Li formulated for extended release, optionally (b) a container that contains a composition that includes a second therapeutic agent, and optionally (c) informational material. The informational material can be descriptive, instructional, marketing or other material that relates to the methods described herein and/or the use of the agents for therapeutic benefit. In one embodiment, the kit also includes a second agent. For example, the kit includes a first container that contains a composition that includes a composition that includes 6-Li formulated for extended release, and a second container that includes the second agent, such as an agent to treat bipolar disorder, or a psychotic symptom.

The informational material of the kits is not limited in its form. In one embodiment, the informational material can include dosing information, how frequently the composition should be administered, formulation, lithium (e.g., 6-Li) concentration, date of expiration, batch or production site information, and so forth. In one embodiment, the informational material relates to methods of administering the 6-Li composition, e.g., in a suitable dose, dosage form, frequency of administration (e.g., a dose, dosage form, or frequency of administration described herein), to treat a subject who has bipolar disorder, mania, or other psychotic disorders. The information can be provided in a variety of formats, including printed text, computer readable material, video recording, or audio recording, or information that provides a link or address to substantive material.

In addition to 6-Li, the pharmaceutical composition provided in the kit can include other ingredients, such as a solvent or buffer, a stabilizer, or a preservative. The composition can be provided in any form, e.g., a liquid, dried or lyophilized form, and substantially pure and/or sterile. Typically, the composition is in an extended release form, e.g., in a tablet or capsule.

A kit can include one or more containers for the composition or compositions containing the 6-Li. In some embodiments, the kit contains separate containers, dividers or compartments for the composition and informational material. For example, the composition can be contained in a blister pack or bottle, and the informational material can be contained in a plastic sleeve or packet. In other embodiments, the separate elements of the kit are contained within a single, undivided container. For example, the composition is contained in a blister pack or bottle that has attached thereto the informational material in the form of a label. In some embodiments, the kit includes a plurality (e.g., a pack) of individual containers, each containing one or more unit dosage forms (e.g., a dosage form described herein) of the agents. The containers can include a combination unit dosage, e.g., a unit that includes both the 6-Li composition and the second agent, such as in a desired ratio. For example, the kit can include a plurality of foil packets, blister packs, or medical devices each containing, for example, a single combination unit dose. The containers of the kits can be air tight, waterproof (e.g., impermeable to changes in moisture or evaporation), and/or light-tight.

The invention is further illustrated by the following example, which should not be construed as further limiting.

### EXAMPLE

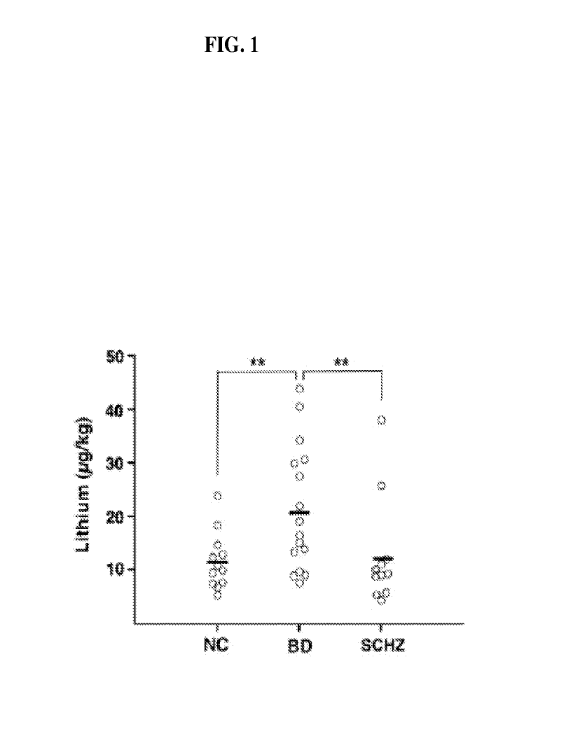

Lithium (Li) isotope levels have been measured in post-mortem neocortex (BA7) of human subjects having bipolar disorder, without having bipolar disorder receiving lithium treatment at time of death. Neurologically intact individuals and subjects with schizophrenia were included as controls. Our data showed that total Li levels are elevated in bipolar disorder (FIG. 1)

Subsequent analysis with quadrupole inductively coupled plasma mass spectroscopy shows that in bipolar disorder, lithium is partitioned selectively in the brains of subjects with bipolar. Bipolar brains show a 4% shift away from 7-Li within the total lithium content. Similar fractionation is not seen in schizophrenia or neurologically intact controls. These data provide the first evidence that the stable lithium isoforms not only are differentiated in physiology, but in pathophysiology as well, and furthermore, that one isotope is more significant for disease mechanism than the other.

For the first time, there is a rationale, based in disease mechanism, that the formulation of n-Li carbonate, currently used clinically, can be improved by removing the 7-Li so that a purified formulation of 6-Li may be administered.

### Incorporation by Reference

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### Equivalents

Those skilled in the art will recognize, or be able to ascertain using no more than routine experimentation, many equivalents to the specific embodiments of the invention described herein. Such equivalents are intended to be encompassed by the following claims

We claim:

1. A pharmaceutical composition comprising lithium, wherein the lithium comprises at least 8% 6-Li (e.g., at least 10% 6-Li, at least 20% 6-Li, at least 50% 6-Li, at least 70% 6-Li, at least 80% 6-Li, at least 90% 6-Li, or at least 99% 6-Li); and wherein the composition is formulated for extended release.

2. The composition of claim 1, wherein the composition is in the form of a tablet or capsule.

3. The composition of claim 1, formulated as a salt.

4. The composition of claim 3, wherein the salt is a carbonate, a chloride or an orotate.

5. The composition of claim 1, wherein the composition is formulated for administration for at least 5 days, at least 6 days, at least 7 days, at least one month, or at least one year or longer.

6. The composition of claim 1, wherein the composition is formulated for administration once, twice or three times per day or more.

7. The composition of claim 1, wherein the composition comprises at least 50 mg, at least 100 mg, at least 150 mg, at least 200 mg, at least 250 mg, at least 300 mg, at least 350 mg, at least 400 mg, or at least 450 mg 6-Li.

8. A pharmaceutical composition comprising lithium, wherein the lithium comprises at least 8% 6-Li (e.g., at least 10% 6-Li, at least 20% 6-Li, at least 50% 6-Li, at least 70% 6-Li, at least 80% 6-Li, at least 90% 6-Li, or at least 99% 6-Li); and an additional therapeutic agent.

9. A method of treating a subject comprising administering a composition of claim 1.

10. The method of claim 9, wherein the composition is in the form of a tablet or capsule.

11. The method of claim 9, wherein the composition is formulated as a salt.

12. The method of claim 11, wherein the salt is a carbonate, a chloride or an orotate

13. The method of claim 9, wherein the composition is formulated for administration for at least 5 days, at least 6 days, at least 7 days, at least one month, or at least one year or longer.

14. The method of claim 9, wherein the composition is formulated for administration once, twice or three times per day or more.

15. A method of treating a subject, the method comprising administering to the subject a pharmaceutical composition comprising lithium, wherein the lithium comprises at least 8% 6-Li (e.g., at least 10% 6-Li, at least 20% 6-Li, at least 50% 6-Li, at least 70% 6-Li, at least 80% 6-Li, at least 90% 6-Li, or at least 99% 6-Li) and an additional therapeutic agent.

16. A method of treating a subject, the method comprising administering the subject a pharmaceutical composition comprising lithium, wherein the lithium comprises at least 8% 6-Li (e.g., at least 10% 6-Li, at least 20% 6-Li, at least 50% 6-Li, at least 70% 6-Li, at least 80% 6-Li, at least 90% 6-Li, or at least 99% 6-Li) at an amount sufficient to achieve a therapeutic effect, wherein the composition is administered to the subject for at least about 5 days at least 6 days, at least 7 days, at least one month, or at least one year or longer.
